(19) United States
(12) Patent Application Publication
(10) Pub. No.: US 2014/0219934 A1
(43) Pub. Date: Aug. 7, 2014

(54) BOTANICAL TOOTH WHITENER COMPOSITION AND METHOD FOR TREATING DISCOLORED OR STAINED TEETH

(71) Applicant: Sandra Senzon, New York, NY (US)

(72) Inventor: Sandra Senzon, New York, NY (US)

(21) Appl. No.: 14/160,917

(22) Filed: Jan. 22, 2014

Related U.S. Application Data
(60) Provisional application No. 61/849,702, filed on Feb. 1, 2013.

(51) Int. Cl. A61K 8/97; A61Q 11/00

(52) U.S. Cl. CPC A61K 8/97; A61Q 11/00
USPC 424/58

(57) ABSTRACT

A botanical tooth whitener composition comprises a quantity of camellia oil, a flavoring, and a thickening agent. The composition is comprised of natural substances and is generally free of chemical additives commonly found in related products. The composition may be used to reduce tooth discoloration or staining by brightening the teeth exposed thereto. A method of treating discolored or stained teeth comprises the steps of obtaining a botanical tooth whitener composition, dispensing a quantity thereof onto or into an applicator, and applying the composition to a tooth or teeth via the applicator. The applicator may be any of a spray bottle, brush pen, toothbrush, and dental tray.

### CROSS-REFERENCE TO RELATED APPLICATIONS

The present disclosure claims priority under 35 U.S.C. §119 on pending U.S. Provisional Application Ser. No. 61/849,702, filed on Feb. 1, 2013, the disclosure of which is incorporated by reference.

### FIELD OF THE DISCLOSURE

The present disclosure relates to a botanical tooth whitener composition and apparatus, and, more particularly, to a botanical tooth whitener composition that cosmetically improves the appearance of teeth and gums and an apparatus for delivering the composition to and applying it on a user's teeth and gums.

### BACKGROUND

Proper dental hygiene mandates that an individual regularly visit a dental professional for routine cleaning and otherwise maintain proper care at home as often as three times a day. Caring for one's teeth generally involves brushing them with a cleaning composition such as toothpaste, which may eliminate a substantial amount of bacteria present in the mouth and thus help prevent the user from certain oral health issues such as tooth cavities and the gum disease gingivitis. However, even an individual who routinely cares for his or her teeth and gums may be subjected to other oral issues notwithstanding such care, which other issues may, for example, include tooth discoloration and staining.

Tooth discoloration and staining occurs when certain substances, such as coffee and tea, make contact with the enamel in a person's mouth. Introducing these substances to tooth enamel often results in the tooth enamel at least partially wearing away, and may further irritate or otherwise cause damage to healthy gum tissue. In this way, such materials may have the effect of reducing the brightness or shine conveyed by one's otherwise clean teeth and healthy gums. While tooth discoloration and staining on their own may not pose significant health risks, they may be viewed unfavorably by others and thus may negatively affect one's smile and overall outward appearance.

The widespread and large scale uses of substances having a propensity for discoloring and staining teeth means that more and more people each year are affected thereby. In fact, there has been a greater demand in recent years for products that may reduce or eliminate tooth discoloration and staining. Many of these products may be purchased over the counter at drug and grocery stores and are applied by the user himself or herself over a set period of time. However, these products, while potentially effective at reducing tooth discoloration and staining, may have adverse effects on the user.

In particular, existing solutions in the art utilize chemical compositions comprising peroxides, such as hydrogen peroxide, and/or titanium dioxide, for eliminating or reducing tooth discoloration and staining. Peroxides are compounds made up of oxygen-oxygen chemical bonds and provide a bleaching effect on materials to which they are introduced, which makes them a favored ingredient in existing compositions. Still, many people are allergic or overly sensitive to peroxide formulations, and the use of such formulations by one allergic thereto may result in irritated oral tissues. Further, peroxides may bring about various negative effects even in persons not allergic to them, including the erosion of healthy gum tissue and the stripping of enamel from the surface of teeth. In addition, titanium dioxide, which is used in U.S. Pat. No. 6,231,341, has been reported to cause neurological disease. As such, presently available tooth whitening compositions utilizing peroxides and/or titanium dioxide present issues that may preclude the user from achieving desired dental health.

Thus, there is a need for a tooth whitening composition that achieves the result of reducing tooth discoloration and staining without the use of potentially harmful chemical agents such as peroxides or titanium dioxide. Further, there is a need for a tooth whitening composition that is comprised of materials that do not pose any potential adverse affects to a user's teeth and gums and which therefore do not reduce the user's dental health.

### SUMMARY OF THE INVENTION

In view of the foregoing disadvantages inherent in the prior art, the general purpose of the present disclosure is to provide a botanical tooth whitener composition and apparatus to include all advantages of the prior art, and to overcome the drawbacks inherent in the prior art. It is an object of the present disclosure is to provide a composition that uses natural ingredients to cosmetically improve a user's oral tissue by brightening teeth without irritating or otherwise harming gums, and further to provide a composition that excludes chemicals known to cause dentinal tubular sensitivity and pose other health risks. The present disclosure is also directed towards providing a method for treating discolored or stained teeth using the disclosed composition.

In an embodiment, a botanical tooth whitener composition comprises a quantity of camellia oil, a flavoring, and a thickening agent. The camellia oil is processed from the camellia plant and is free of chemical additives, which camellia oil provides, amongst other things, whitening of and increased shine to tooth enamel. In a further embodiment, the flavoring may be any of a variety of fruit and herbal oils, essences, and extracts (collectively, the “flavoring ingredients”). It will be apparent that the flavoring ingredients may be formulated and combined in various combinations to produce a variety of flavors for the tooth whitener, including, but not necessarily limited to, strawberry, eucalyptus, gummy bear, rosemary, rose hip, marshmallow, cotton candy, apple, cherry, and grape, or any other flavor that may be pleasing to the person that is ingesting the composition. In a further embodiment, the thickening agent may be beeswax. In another embodiment, the thickening agent may be candelilla wax, and in yet another embodiment, the thickening agent may be vegetable glycerin.

In an embodiment, a method for treating discolored or stained teeth comprises the steps of obtaining a botanical tooth whitener composition as disclosed herein, dispensing a quantity thereof onto or into an applicator, and applying the composition to a tooth or teeth via the applicator. In a further embodiment, the applicator may be any of a spray bottle, brush pen, toothbrush, and dental tray.

These together with other aspects of the present disclosure, along with the various features of novelty that characterize the present disclosure, are pointed out with particularity in the claims annexed hereto and form a part of the present disclosure. For a better understanding of the present disclosure, its operating advantages, and the specific objects attained by its uses, reference should be made to the accompanying drawings and detailed description in which there are illustrated and described exemplary embodiments of the present disclosure.

### DESCRIPTION OF THE DRAWINGS

The advantages and features of the present invention will become better understood with reference to the following detailed description and claims taken in conjunction with the accompanying drawings, wherein like elements are identified with like symbols, and in which:

FIG. 1 shows an exemplary diagram of the various items involved in the disclosed method, including the disclosed composition and a plurality of applicators, in accordance with an exemplary embodiment of the present disclosure; and

FIG. 2 shows an exemplary embodiment of the applying step of the disclosed method in accordance with an exemplary embodiment of the present disclosure.

Like reference numerals refer to like parts throughout the description of several views of the drawings.

### DETAILED DESCRIPTION OF THE DISCLOSURE

The best mode for carrying out the present disclosure is presented in terms of its preferred embodiments, herein depicted in the accompanying figures. The preferred embodiments described herein detail for illustrative purposes are subject to many variations. It is understood that various omissions and substitutions of equivalents are contemplated as circumstances may suggest or render expedient, but are intended to cover the application or implementation without departing from the spirit or scope of the present disclosure.

The terms “a” and “an” herein do not denote a limitation of quantity, but rather denote the presence of at least one of the referenced items.

The present disclosure provides a botanical tooth whitener composition for reducing tooth discoloration or staining. In an embodiment, the composition is comprised of a quantity of camellia oil, a flavoring agent, and a thickening agent. The camellia oil provides, amongst other things, whitening of and increased shine to tooth enamel. The flavoring may be any of a variety of fruit and herbal oils, essences, and extracts, which contribute a pleasant or otherwise neutral taste to the composition for the user's benefit. The thickening agent may be any substance capable of increasing the viscosity of the composition, which, in an embodiment, is beeswax. In another embodiment, the thickening agent may be candelilla wax, and in yet another embodiment, the thickening agent may be vegetable glycerin. The composition is specifically free of chemical additives, such as peroxides or titanium dioxide, which omission precludes the composition from presenting adverse effects to a user's oral tissues through its use. The composition may be used by an individual to reduce any tooth discoloration or stating caused by the introduction of various discoloring substances thereto.

The present disclosure further provides a method for treating discolored or stained teeth using the botanical tooth whitener composition discussed herein. In an embodiment, the disclosed method comprises the steps of obtaining a botanical tooth whitener composition, dispensing a quantity thereof onto or into an applicator, and applying the composition to a tooth or teeth via the applicator. The applicator may be any of a spray bottle, brush pen, toothbrush, and dental tray. A user may follow the steps of the disclosed method in order to reduce any discoloration or staining present on his or her teeth.

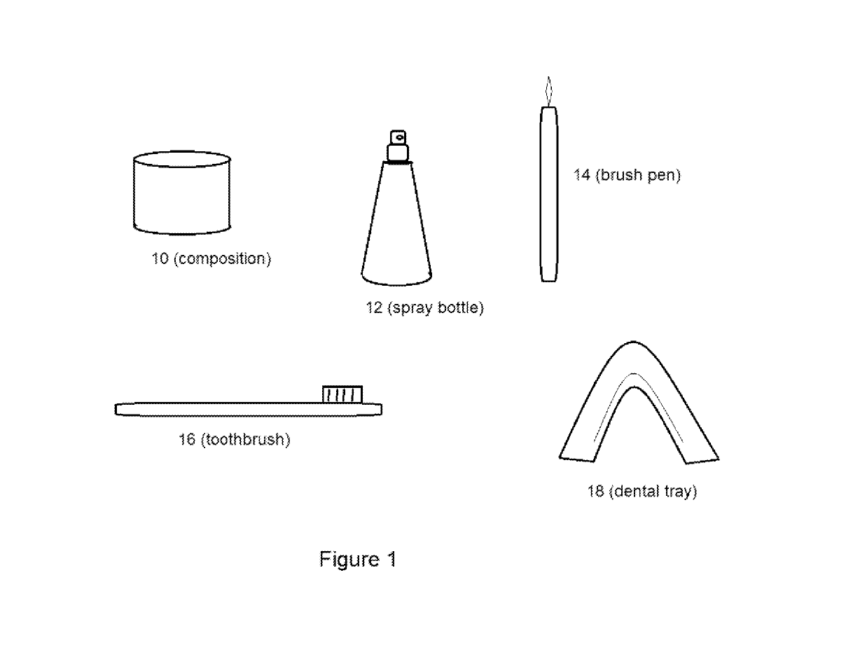

Referring now to FIG. 1, a botanical tooth whitener composition is shown in an exemplary container at 10. The composition 10 is a liquid that may vary in viscosity depending on the quantity of thickening agent included therein. For example, where only a modicum of thickening agent is used, the composition may be a thin liquid, and where a significant amount of thickening agent is used, the composition may have a generally viscous quality. The main ingredient of the composition 10 is camellia oil, and it will be apparent that a majority of the composition 10 may thus comprise camellia oil. Camellia oil is processed from the seed of the flowers from Camellia, a plant genus within which certain shrubs and plants are classified, and camellia oil provides, amongst other things, whitening of and increased shine to tooth enamel. Camellia flowers are commonly found in eastern and southern Asia and are present on those plants that are used to make white, green, and black teas.

The composition 10 is formulated by using a particular amount of camellia oil in conjunction with a flavoring and a thickening agent. Namely, to make the composition, the flavoring and thickening agent are introduced to the quantity of camellia oil being used, which introduction creates a porous mass that is thereafter drained of extra, unnecessary moisture. As stated above, the specific viscosity of the composition 10 may vary based on the amount of thickening agent included therein. In a preferred embodiment, the quantity of thickening agent used within the composition 10 is equal to seven percent of the quantity of camellia oil. However, it will be apparent that the composition 10 will sufficiently reduce tooth discoloration or staining regardless of the particular quantity of thickening agent included therein. In an embodiment, the thickening agent used within the composition 10 may be beeswax. In another embodiment, the thickening agent may be candelilla wax, and in yet another embodiment, the thickening agent may be vegetable glycerin.

In an embodiment, the composition 10 may be incorporated into a paste such as a toothpaste. In such an embodiment, the flavoring may comprise xylitol, sorbitol, or other suitable ingredient for flavoring a toothpaste. In this embodiment, the thickening agent may be xanthan gum. It will be apparent that the toothpaste that incorporates the composition 10 may be formulated with other additional natural and/or non-harmful ingredients that may increase the efficacy of the toothpaste and/or may be necessary or customary to formulate a toothpaste otherwise known in the art, without deviating from the spirit of the claimed disclosure.

The flavoring is a substance that provides a pleasant or otherwise neutral taste to the composition 10 for the user's enjoyment or benefit. In an embodiment, the flavoring is comprised of oils, essences, or extracts prepared from one or more fruits or herbs, such as peppermint oil, lavender oil, spearmint oil, clove oil, citrus oils, fruit essences, mint, anise, sage, strawberry, apple, banana, watermelon, papaya, and orange. It will be apparent that the aforementioned list of flavorings is non-exhaustive and that other flavorings may be used with the composition 10. In an embodiment, the quantity of flavoring used within the composition 10 is equal to one drop for each included flavor.

The use of the composition 10 creates the appearance of brighter teeth establishing tooth surface light refraction, which refraction provides for a greater shine on the teeth without any change being made to the particular shades thereof. The light refraction properties are founded in the camellia oil's naturally high level of anti-oxidants, and may have the effect of reducing the appearance of tooth discoloration or staining by imposing an even shine across the surface of the teeth. The composition 10 has a refractive index of over 2.49, which is higher than that of many peroxide-based formulations, thereby making it a desirable alternative to existing stain removal compositions on the market.

As will be discussed below, the composition may be applied to the user's teeth in any of a plurality of ways. For example, the user may use an applicator to apply the composition to the discolored or stained teeth in his or her mouth. The various applicators are depicted in FIG. 1 as being a spray bottle 12, a brush pen 14, a toothbrush 16, and a dental tray 18. As will be discussed below, the user may dispense a quantity or portion of the composition onto or into the chosen applicator in order to apply it to his or her teeth. In an alternate embodiment, the composition 10 may be used as a substitute or supplement for a mouthwash. Thus, a user may use the composition 10 to effectively brighten the surface of his or her teeth by swishing the composition 10 within his or her mouth for a set duration of time. Further, using the composition 10 as a mouthwash substitute or supplement may provide additional health benefits for the user, such as cleansing the mouth and body by removing potentially harmful toxins therefrom.

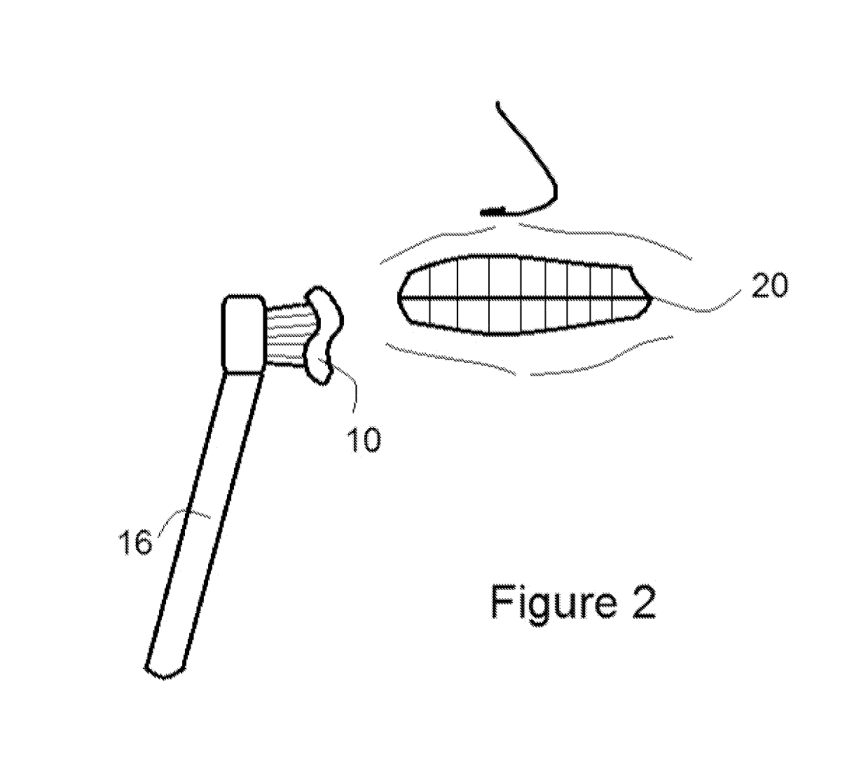

A step of a method for treating discolored or stained teeth is shown in FIG. 2. The method first requires a user 20 to obtain a botanical tooth whitener composition 10 such as that which is described above, which composition 10 may be stored within a jar, tube, or other container prior to beginning the method. The user 20 may then dispense a quantity or portion of the composition 10 from the container on which it is disposed onto an applicator, which applicator, as previously mentioned, may be any of a spray bottle, brush pen, toothbrush, and dental tray. It will be apparent that any applicator capable of applying the composition 10 onto a tooth or teeth may be sufficient for use with the disclosed method. The user 20 may determine the desired quantity to dispense onto the applicator based on the number of teeth he or she wishes to brighten.

The user 20 applies the composition 10 to his or her teeth using the applicator, such as a toothbrush 16. The user 20 may spread the composition 10 about his or her oral tissue using the applicator for full effect. The result of such application is a noticeable improvement to the brightness and cleanliness of the user's 20 teeth. Specifically, the teeth exposed to the composition 10 will appear significantly brighter than they had previously due to the light refracting qualities of the camellia oil comprised within the composition 10. The user 20 may use the composition 10 in this way to reduce the level of discoloration or staining on his or her teeth multiple times each day without facing any adverse effects therefrom due to the chemical-free nature of the composition 10.

The high levels of peroxides in existing stain remover compositions may cause a user's gum tissue to become irritated when coming into contact therewith. As such, users of such existing stain removers are generally required or otherwise recommended to place a barrier between their gums and teeth or otherwise ensure that such stain removers are precluded from contacting their gums. However, by excluding peroxides and similar chemicals therefrom, the disclosed composition 10 does not pose any health risks to a user's gum tissue. Thus, unlike existing methods for treating tooth discoloration or staining using existing stain removers, the disclosed method does not require the user to protect his or her gums from being contacted by the disclosed composition 10. In fact, the anti-oxidant qualities about the included camellia oil may even help to reduce existing inflammation on or irritation of the user's gum tissue.

The present disclosure offers the following advantages. The composition brightens discolored or stained teeth using natural ingredients, thereby reducing tooth discoloration or staining without the need for chemical additives that may otherwise negatively affect the user's oral tissues. The method discloses a convenient way to use the disclosed composition using a variety of applicators and, due to the natural and chemical-free nature of the composition, may be repeated multiple times a day without subjecting the user to any dental health risks. The composition may be used by both the young and the elderly, and may safely be used as often as three to five times each day.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The exemplary embodiment was chosen and described in order to best explain the principles of the present disclosure and its practical application, to thereby enable others skilled in the art to best utilize the disclosure and various embodiments with various modifications as are suited to the particular use contemplated.

What is claimed is:

1. A botanical tooth whitener composition, the composition comprising:
between 0.01% and 99.99% camellia oil;
between 0.01% and 99.99% flavoring; and
between 0.01% and 99.99% thickening agent.

2. The composition as claimed in claim 1, wherein said flavoring comprises one or more of peppermint oil, lavender oil, spearmint oil, clove oil, citrus oils, xylitol, sorbitol, fruit essences, mint, anise, sage, strawberry, apple, banana, watermelon, papaya, and orange.

3. The composition as claimed in claim 1, wherein said thickening agent is beeswax.

4. The composition as claimed in claim 1, wherein said thickening agent is candelilla wax.

5. The composition as claimed in claim 1, wherein said thickening agent is vegetable glycerin.

6. The composition as claimed in claim 1, wherein said thickening agent is xanthan gum.

7. A method of treating discolored or stained teeth, the method comprising the steps of:
obtaining a botanical tooth whitener composition, wherein the composition comprises:
between 0.01% and 99.99% camellia oil;
between 0.01% and 99.99% flavoring; and
between 0.01% and 99.99% thickening agent;
dispensing a quantity of the composition onto or into an applicator; and
applying the composition to one or more teeth via the applicator.

8. The method as claimed in claim 7, wherein the flavoring of the composition comprises one or more of peppermint oil, lavender oil, spearmint oil, clove oil, citrus oils, xylitol, sorbitol, fruit essences, mint, anise, sage, strawberry, apple, banana, watermelon, papaya, and orange.

9. The method as claimed in claim 7, wherein the thickening agent of the composition is beeswax.

10. The method as claimed in claim 7, wherein said thickening agent is candelilla wax.

11. The method as claimed in claim 7, wherein said thickening agent is vegetable glycerin.

12. The method as claimed in claim 7, wherein the applicator is a spray bottle.

13. The method as claimed in claim 7, wherein the applicator is a brush pen.

14. The method as claimed in claim 7, wherein the applicator is a toothbrush.

15. The method as claimed in claim 7, wherein the applicator is a dental tray.

16. The composition as claimed in claim 7, wherein said thickening agent is xanthan gum.
